(19) United States
(12) Patent Application Publication
(10) Pub. No.: US 2026/0062735 A1
(43) Pub. Date: Mar. 5, 2026

(54) LYOPHILIZED OR LYOPHILIZABLE INDICATOR MASTER MIX FOR NUCLEIC ACID AMPLIFICATION AND VISUAL DETECTION OF A BIOLOGICAL TARGET

(71) Applicant: Definitive Biotechnologies LLC, Elkridge, MD (US)

(72) Inventors: Timothy Alcorn, Raleigh, NC (US); Peter Latham, Action, MA (US); Todd Haran, Bloomfield, NY (US); Scott Rosebrough, Avon, NY (US)

(21) Appl. No.: 19/313,270

(22) Filed: Aug. 28, 2025

Related U.S. Application Data
(60) Provisional application No. 63/688,063, filed on Aug. 28, 2024.

(51) Int. Cl. C12Q 1/6806

(52) U.S. Cl. CPC C12Q 1/6806

(57) ABSTRACT

Indicator master mix for performing nucleic acid amplification assays to detect and visually identify the presence or absence of target or template DNA or RNA sequences. The indicator master mix comprises (i) a human readable, substantially hydrophobic nucleic-acid based indicator and (ii) a solvent configured to solubilize the substantially hydrophobic nucleic-acid based indicator, such as DMSO. The relative amount of nucleic-acid based indicator to solvent is within the range of (i) about 0.01 gm nucleic-acid based indictor/gm solvent to (ii) about 0.05 gm nucleic-acid based indicator/gm solvent. The master mix is configured to perform the nucleic acid amplification assay to detect and visually identify the presence or absence of the target or template DNA or RNA sequence, and the substantially hydrophobic nucleic-acid based indicator is visually detectable by a human eye to indicate the presence or absence of the target or template DNA or RNA sequence.

### CROSS REFERENCE TO RELATED APPLICATIONS

This application claims benefit under 35 U.S.C. § 119(e) to co-pending U.S. provisional application No. 63/688,063 filed on Aug. 28, 2024, entitled “Lyophilized or Lyophilizable Indicator Master Mix for Nucleic Acid Amplification and Visual Detection of a Biological Target,” which is hereby incorporated by reference in its entirety as if fully set forth herein.

### FIELD OF THE INVENTION

The present disclosure relates to nucleic acid amplification assays for detecting the presence or the absence of target or template DNA or RNA sequences in biological samples, and more particularly, to master mixes with indicators for performing such nucleic acid amplification assays and to related methods of making and using them.

### BACKGROUND INFORMATION

Master mixes are used in Polymerase Chain Reaction (PCR) and Loop-Mediated Isothermal Amplification (LAMP) nucleic acid amplification techniques or processes for detecting the presence or the absence of target or template DNA or RNA sequences in biological samples. A master mix, which is sometimes known as a super mix or ready mix, typically contains a mixture of precursors and enzymes necessary to the PCR or LAMP technique. A lyophilized master mix is advantageous because it has a relatively long shelf life, and therefore can be stored dry, such as in a kit or testing device, and later combined with reagents and a biological sample, such as blood or saliva, to perform a nucleic acid amplification assay to detect the presence (or lack thereof) of target DNA/RNA in the biological sample.

A popular visually detectable agent or indicator used in LAMP and PCR master mixes is Calcein. Calcein is a fluorescent dye that is visually detectable with a human eye under fluorescent light. The prior art is not believed to have successfully lyophilized Calcein-containing LAMP-based or PCR-based solutions. Such indicator master mixes are water based, and a solvent, such as dimethyl sulfoxide (“DMSO”), is typically used to solubilize the Calcein in water. One problem with DMSO is that it prevents the resulting solution from being lyophilized. Thus, while DMSO can effectively solubilize Calcein in water-based solutions, such solutions have not been lyophilizable. Rather, efforts to lyophilize known DMSO-containing master mixes have resulted in a “sludge” like substance.

It is an object of the present invention, and/or of embodiments thereof, to overcome one or more of the above-described drawbacks and/or disadvantages of the prior art.

### SUMMARY OF THE INVENTION

In accordance with one aspect, the present invention is directed to an indicator master mix for performing a nucleic acid amplification assay to detect and visually identify the presence or the absence of a target or template DNA or RNA sequence. The master mix comprises (i) a human readable, substantially hydrophobic nucleic-acid based indicator, and (ii) a solvent configured to solubilize the substantially hydrophobic nucleic-acid based indicator. The relative amount of nucleic-acid based indicator to solvent is within the range of (i) about 0.01 gm nucleic-acid based indictor/gm solvent to (ii) about 0.05 gm nucleic-acid based indicator/gm solvent. The indicator master mix is configured to perform the nucleic acid amplification assay to detect and visually identify the presence or the absence of the target or template DNA or RNA sequence, and the substantially hydrophobic nucleic-acid based indicator is visually detectable by a human eye to indicate the presence or absence of the target or template DNA or RNA sequence. In some embodiments, the human readable, substantially hydrophobic nucleic-acid based indicator is configured to react to one or more diprotic cations to detect and visually identify the presence or absence of the target or template DNA or RNA sequence.

In some embodiments of the present invention, the human readable, substantially hydrophobic nucleic-acid based indicator is isothermal. In some embodiments, the relative amount of nucleic-acid based indicator to solvent is within the range of (i) about 0.009 gm nucleic-acid based indictor/gm solvent to (ii) about 0.045 gm nucleic-acid based indicator/gm solvent. In some embodiments, the human readable, substantially hydrophobic nucleic-acid based indicator is quenched Calcein. In some such embodiments, the solvent is an aprotic solvent. In some such embodiments, the aprotic solvent is selected from the group including: Acetone, Acetonitrile, DMF (N,N-Dimethylformamide), DMSO (Dimethyl sulfoxide), DCM (Dichloromethane), THF (Tetrahydrofuran), HMF (hydroxymethylfurfural) and Crown ethers. In some such embodiments, the aprotic solvent is DMSO.

In some embodiments of the present invention, the solvent is configured to solubilize the substantially hydrophobic nucleic-acid based indicator into an aqueous-based solution of the master mix. In some embodiments, the indicator master mix is substantially dehydrated and defines a form factor configured to perform a nucleic acid amplification assay on a respective biological sample to detect and visually identify the presence or absence of the target or template DNA or RNA sequence in the respective biological sample. In some such embodiments, the indicator master mix is lyophilized. In some such embodiments, the indicator master mix is formed into a plurality of lyobeads, and one or more of the lyobeads are configured to perform a nucleic acid amplification assay on a respective biological sample.

In accordance with another aspect, the present invention is directed to an indicator master mix for performing a nucleic acid amplification assay to detect and visually identify a presence or an absence of a target or template DNA or RNA sequence in a biological sample. The indicator master mix comprises: (i) first substantially hydrophobic means for detecting and visually identifying with a human eye the presence or the absence of the target or template DNA or RNA sequence in the biological sample; and (ii) second means for solubilizing the first means. The relative amount of first means to second means is within the range of (i) about 0.01 gm first means/gm second means to (ii) about 0.05 gm first means/gm second means. The indicator master mix is configured to perform the nucleic acid amplification assay to detect and visually identify the presence or the absence of the target or template DNA or RNA sequence in the biological sample. The first means is visually detectable by the human eye to indicate the presence or the absence of the target or template DNA or RNA sequence in the biological sample.

In some embodiments of the present invention, the first means is a human readable, substantially hydrophobic nucleic-acid based indicator, and the second means is a solvent configured to solubilize the substantially hydrophobic nucleic-acid based indicator. In some such embodiments, the human readable, substantially hydrophobic nucleic-acid based indicator is quenched Calcein, and the solvent is an aprotic solvent selected from the group including: Acetone, Acetonitrile, DMF (N,N-Dimethylformamide), DMSO (Dimethyl sulfoxide), DCM (Dichloromethane), THF (Tetrahydrofuran), HMF (hydroxymethylfurfural) and Crown ethers.

In accordance with another aspect, the present invention is directed to a method comprising the following steps:
solubilizing a human readable, substantially hydrophobic nucleic-acid based indicator in a solvent, wherein the relative amount of nucleic-acid based indicator to solvent is within the range of (i) about 0.01 gm nucleic-acid based indictor/gm solvent to (ii) about 0.05 gm nucleic-acid based indicator/gm solvent;
combining the nucleic-acid based indicator and solvent solution with an aqueous-based solution containing a cofactor or diprotic cation containing compound and quenching the nucleic-acid based indicator in the solution; and
lyophilizing the quenched nucleic-acid based indicator solution and forming a lyophilized indicator master mix, wherein the relative amount of nucleic-acid based indicator to solvent in the lyophilized indicator master mix is within the range of (i) about 0.01 gm nucleic-acid based indictor/gm solvent to (ii) about 0.05 gm nucleic-acid based indicator/gm solvent.

In some embodiments of the present invention, the method further comprises, prior to the step of lyophilizing, adding to the quenched nucleic-acid based indicator solution one or more enzymes, nucleotides, buffers, stabilizers, enhancers, primers, probes and/or excipients. In some such embodiments, the method further comprises aliquoting the indicator master mix into a plurality of reaction vessels prior to the lyophilizing step. In some such embodiments, the reaction vessels are reaction tubes, and at least a portion of each reaction tube is transparent or substantially transparent to enable visualization therethrough by a human eye to visually observe the presence or absence of the target or template DNA or RNA sequence. In some embodiments of the present invention, the human readable, substantially hydrophobic nucleic-acid based indicator is Calcein, and the cofactor or diprotic cation containing compound is manganese chloride (MnCl₂).

In accordance with another aspect, the present invention is directed to a method comprising the following steps:

hydrating a lyophilized indicator master mix with a processed biological sample, wherein the lyophilized indicator master mix contains a quenched nucleic-acid based indicator and a solvent, and the relative amount of nucleic-acid based indicator to solvent is within the range of (i) about 0.01 gm nucleic-acid based indictor/gm solvent to (ii) about 0.05 gm nucleic-acid based indicator/gm solvent; and
visually observing the appearance of the hydrated indicator master mix and biological sample with a human eye and detecting with the human eye the presence or an absence of a target or template DNA or RNA sequence.

In some embodiments of the present invention, the method further comprises incubating the hydrated indicator master mix and biological sample at a substantially constant temperature for a substantially predetermined period of time prior to visually observing the appearance of the hydrated indicator master mix and biological sample, and then visually detecting the presence or the absence of the target or template DNA or RNA sequence. In some such embodiments, the human readable, substantially hydrophobic nucleic-acid based indicator is Calcein, and the solvent is selected from the group including Acetone, Acetonitrile, DMF (N,N-Dimethylformamide), DMSO (Dimethyl sulfoxide), DCM (Dichloromethane), THF (Tetrahydrofuran), HMF (hydroxymethylfurfural) and/or Crown ethers.

One advantage of the present invention, and/or of one or more embodiments thereof, is that it provides a lyophilizable or lyophilized indicator master mix, such as a LAMP or PCR indicator master mix, that contains a substantially hydrophobic nucleic-acid based indicator, such as Calcein. Accordingly, the present invention, and/or one or more embodiments thereof, overcomes the above-described problem in the prior art where Calcein-containing master mixes have not been lyophilizable or lyophilized.

Another advantage of the present invention, and/or of one or more embodiments thereof, is that it enables an indicator master mix, such as a LAMP or PCR master mix, to be lyophilizable or lyophilized, and further, to include a detectable indicator or agent that is visibly detectable by the human eye.

Another advantage of the present invention, and/or of one or more embodiments thereof, is in the use of Calcein or other substantially hydrophobic nucleic-acid based indicators. In contrast to the above-described prior art, the Calcein or other substantially hydrophobic nucleic-acid based indicator is lyophilizable or is lyophilized and provides a human readable feedback mechanism that does not require additional instrumentation to interpret the results of the assay. Other lyophilizable indicators are not human readable and, instead, require additional instrumentation to interpret the results of the assay.

Another advantage of the present invention, and/or of one or more embodiments thereof, is the provision of the indicator master mix in a convenient, lyophilized form factor, such as a lyobead. Lyobeads can be easily handled and have greater than a two year shelf life which allows clinical labs to store them in inventory for use as needed. The indicator master mix of the present disclosure provides such labs with a commercial advantage as it makes testing more efficient and cost-effective enabling labs to perform widespread screening for disease and other applications, such as fetal gender identification. In the device space, due to the long shelf life, human readable characteristics and manufacturing benefits of a solid sphere or similar form factor, such as a lyobead, over multiply solutions, the fully integrated lyobead lends itself to POND (Point of Need Diagnostics) molecular diagnostic devices that are cost-effective, simple to use and field deployable.

Other advantages of the present invention, and/or of one or more embodiments thereof, will become more readily apparent in view of the following detailed description and accompanying drawings.

### BRIEF DESCRIPTION OF THE DRAWINGS

FIG. 1 is a flowchart illustrating the procedural steps for preparing an indicator master mix where the form factor of the master mix is a lyobead and a plurality of such lyobeads are prepared; and

FIG. 2 is a flowchart including the procedural steps of FIG. 1 for preparing the indicator master mix in the form of a plurality of lyobeads, and further illustrating the steps for rehydrating one or more lyobeads with one or more respective processed biological samples, incubating the rehydrated master mixes and samples, and then viewing with a human eye the resulting mixtures to detect the presence or the absence of target or template DNA or RNA sequences in the biological samples.

### DETAILED DESCRIPTION OF EMBODIMENTS

The present disclosure is directed to a human readable and lyophilizable PCR or LAMP solution that contains the reagents required to perform a molecular assay that can be used to detect and visually identify the presence or absence of a target (or template) DNA or RNA sequence. Often referred to as a “master mix,” and depending on the application, the solution contains multiple ingredients, such as enzymes, nucleotides, primers, a buffer solution, stabilizers and an indicator chemistry with the function to provide user feedback on the presence or the lack thereof of a target DNA or RNA sequence.

When lyophilizing an indicator master mix of the present disclosure, the ingredients are mixed with an additive called an excipient. Excipient(s) help to stabilize the physical structure of the master mix in its final form, which is often a substantially spherical or bead shape called a “lyobead.” However, the final form of the indicator master mix can take other shapes that are currently known or that later become known, or can be dried as a powder as well. The solution preferably is aliquoted into single reaction volumes and freeze dried (or lyophilized) into its final form factor.

In molecular assays, a specific DNA or RNA sequence that is unique to a target virus, bacteria, chromosome, etc. is replicated exponentially during the assay. Each ingredient of the indicator master mix performs a specific function in connection with or during the molecular assay. PCR and LAMP based molecular assays require indicator master mixes unique to the respective process or technique. They differ in specific areas, but are quite similar in many ways. One similarity is the need for an indicator to perform the final function of the assay, which is to indicate the results of the tests to users.

Typical master mix ingredients for both PCR and LAMP based molecular assays include the following:

Enzymes, including DNA or RNA polymerase enzymes: These are the enzymes responsible for synthesizing new DNA or RNA strands from the template nucleic acid during the amplification process. They are used in PCR and LAMP assays

Nucleotides (dNTPs or NTPs): These are the building blocks required for the synthesis of new DNA or RNA strands. These include adenine (A), cytosine (C), guanine (G), and thymine (T) and/or uracil (U).

Buffer solution: This is a buffer system configured to maintain the desired, predetermined or optimal pH and ionic conditions for the enzymatic reaction.

Stabilizers and enhancers: These may include additives to improve enzyme stability, enhance amplification efficiency, and/or to prevent non-specific reactions.

Primers and probes: These are relatively short nucleic acid sequences designed to anneal specifically to the target region of interest. Probes may be included for assays such as quantitative PCR (qPCR) or real-time PCR. They are unique to the target of interest as every living species has a unique set.

Excipients: In lyophilization, an excipient refers to any substance added to the product that is not the active but serves a specific purpose in the formulation process or the final product. Excipients play various roles, such as physical stabilization, improving solubility, enhancing bioavailability, or aiding in the manufacturing process. Excipients are often added to the formulation to provide stability to the product during freeze-drying and to improve its reconstitution properties after rehydration.

Indicator: An indicator is a molecule, compound, or substance that is used to detect the presence, absence and/or quantity of a specific target molecule or analyte of interest. In nucleic acid-based diagnostics, like PCR and LAMP, indicators might include fluorescent dyes or molecular probes that bind specifically to the target nucleic acid sequence. When these indicators bind to the target molecule, they produce a detectable signal, such as fluorescence, which can be measured to determine the presence and quantity of the target. Indicators are not only fluorescent. In some instances, indicators are configured to trigger a color change or a change in turbidity to indicate results. Indicators are not always activated by specific molecular binding. Some indicators are triggered by a change in the PH of the assay. Fluorescent indicators include the following:

Fluorescent dyes: Fluorescent dyes, such as SYBR Green, EvaGreen, or Calcein, are commonly used in nucleic acid-based assays like PCR or qPCR. These dyes intercalate into double-stranded DNA and emit fluorescence upon binding, allowing for the real-time monitoring of DNA amplification.

Fluorescent probes: Fluorescent probes, such as TaqMan probes, molecular beacons, or Scorpion probes, are specific oligonucleotide sequences labeled with a fluorophore and a quencher. They hybridize to the target sequence and emit fluorescence when the probe is cleaved or undergoes a conformational change due to polymerase activity.

Chemiluminescent substrates: Chemiluminescent substrates, like luminol or acridinium esters, produce light upon enzymatic reaction. They are often used in immunoassays and nucleic acid detection assays for sensitive detection of target molecules.

Colorimetric indicators: Colorimetric indicators produce a visible color change upon interaction with the target molecule. Examples include dyes, such as bromophenol blue, phenol red, or nitro blue tetrazolium (NBT), which are used in various assays for qualitative or semi-quantitative detection.

Electrochemical indicators: Electrochemical indicators measure changes in electrical signals resulting from the interaction between the target molecule and specific electrodes. This technique is used in biosensors for rapid and sensitive detection of analytes.

Radioactive indicators: Although less common due to safety concerns, radioactive isotopes like 32P or 35S can be used as indicators in molecular diagnostics for highly sensitive detection of target molecules through autoradiography or scintillation counting.

In accordance with one embodiment, the following procedure is employed to prepare solutions of 40× Calcein and 40× Quenched Calcein. The term “quenched” is used herein to describe the state of Calcein (or other indicator) in which the Calcein is bound to MnCl₂ (or other diprotic cation containing compound) in solution and is not fluorescent. The 40× Quenched Calcein is used as the visual indicator for LAMP reactions and the 40× Calcein is used as a control as hereinafter described.

The following ingredients are used to prepare the 40× Calcein and 40× Quenched Calcein solutions:
Calcein: As indicated above, Calcein is a fluorescent dye (molecular weight: 622.55 g/mol, CAS-No 154071-48-4, supplied by Millipore). Although Calcein is the indicator used in the disclosed embodiment, numerous other indicators or dyes that are hydrophobic or marginally soluble in water that are currently known, or that later become known, equally may be employed;
MnCl₂: Manganese (II) chloride tetrahydrate (molecular weight: 197.91 g/mol, CAS-No 13446-34-9, supplied by Sigma Aldrich);
DMSO: Dimethyl sulfoxide. Although DMSO is the solvent used in the currently disclosed embodiment, numerous other solvents that are currently known, or that later become known, equally may be employed, including without limitation Acetone, Acetonitrile, DMF (N,N-Dimethylformamide), DCM (Dichloromethane), THF (Tetrahydrofuran), HMF (hydroxymethylfurfural) and/or Crown ethers;
RNAse free water; and
5 mM Tris Buffer (pH=8).

The following materials may be collected prior to starting the procedure:

Reagents: Calcein, Manganese (II) chloride tetrahydrate (MnCl₂), DMSO and RNAse Free Water, and 5 mM Tris Buffer (pH=about 8).

Instruments/Supplies: A calibrated electronic balance, a small weighing spatula, a small beaker (about 20 milliliters (mL)), a conical tube (about 15 mL), additional conical tubes (about 1.7 mL), a pipette (about 100 microliters (uL)) plus tips, a larger pipette (about 1000 uL) plus tips, and a Qubit 4 Fluorometer. The user should wear gloves when preparing Calcein and MnCl₂ solutions as hereinafter set forth.

A 50 mM Calcein solution is formulated in accordance with the following steps and instructions:

| Steps | Instructions |
|---|---|
| 1 | Tare the electronic balance with a 1.7 mL tube inside a small |
| | holder. |
| 2 | Using a small weighing spatula, weigh out approximately 6 |
| | mg of Calcein into the 1.7 mL tube. |
| 3 | Calculate the necessary volume of DMSO to bring the solution |
| | to a 50 mM concentration (for 6 mg it is 0.193 mL). |
| 4 | Add the calculated volume of DMSO to the 1.7 mL tube. |
| 5 | Mix until the Calcein is completely dissolved (it may take |
| | about 15 minutes for the Calcein to go into solution). |

A 20.4 mM MnCl₂ solution is formulated in accordance with the following steps and instructions:

| Steps | Instructions |
|---|---|
| 1 | Tare the electronic balance with a 15 mL tube inside a |
| | small beaker. |
| 2 | Using a small weighing spatula, weigh out about 28 mg |
| | of MnCl₂ into the 15 mL tube. |
| 3 | Calculate the necessary volume of RNAse free water to |
| | bring the solution to 20.4 mM concentration (for 28 mg |
| | it is about 6.94 mL). |
| 4 | Add the calculated volume of water to the 15 mL tube. |
| 5 | Mix the solution until MnCl₂ is dissolved. |

The 40× Quenched Calcein solution is prepared by combining the 50 mM Calcein and 20.4 mM MnCl₂ solutions as follows:

| Solution | Volume |
|---|---|
| 50 mM Calcein | 10 uL |
| 20.4 mM MnCl₂ | 490 uL |
| TOTAL VOLUME | 500 uL |

The 40× Calcein solution is prepared by combining the 50 mM Calcein solution and RNAse Free Water as follows:

| Solution | Volume |
|---|---|
| 50 mM Calcein | 10 uL |
| RNAse Free Water | 490 uL |
| TOTAL VOLUME | 500 uL |

Greater or lesser volumes of each solution may be prepared, as needed, by proportionally increasing or decreasing the amounts of the respective ingredients, as set forth above.

A quality control check may be performed as follows:

Introduce a 40× dilution of Calcein and Quenched Calcein into the 5 mM Tris pH 8.0 and read with the Qubit Fluorometer. For example:

Dilute 1.25 uL 40× Calcein into 48.75 uL 5 mM Tris buffer in a PCR tube and mix.

Dilute 1.25 uL 40× Quenched Calcein into 48.75 uL 5 mM Tris buffer in a PCR tube and mix.

Illuminate each solution in the respective tube using a blue wavelength LED and observe visually. Calcein should have green fluorescence. Quenched Calcein, on the other hand, should not have fluorescence.

Dilute 10 uL each of these solutions into 190 uL 5 mM Tris buffer and read in the Qubit 4 Fluorometer. Calcein should have a relative fluorescence of about 100,000 whereas Quenched Calcein should have a low reading of about 30% to 40% or less of the Calcein reading, e.g., about 300. The 100,000 value approximates the highest amount of fluorescence for a LAMP assay.

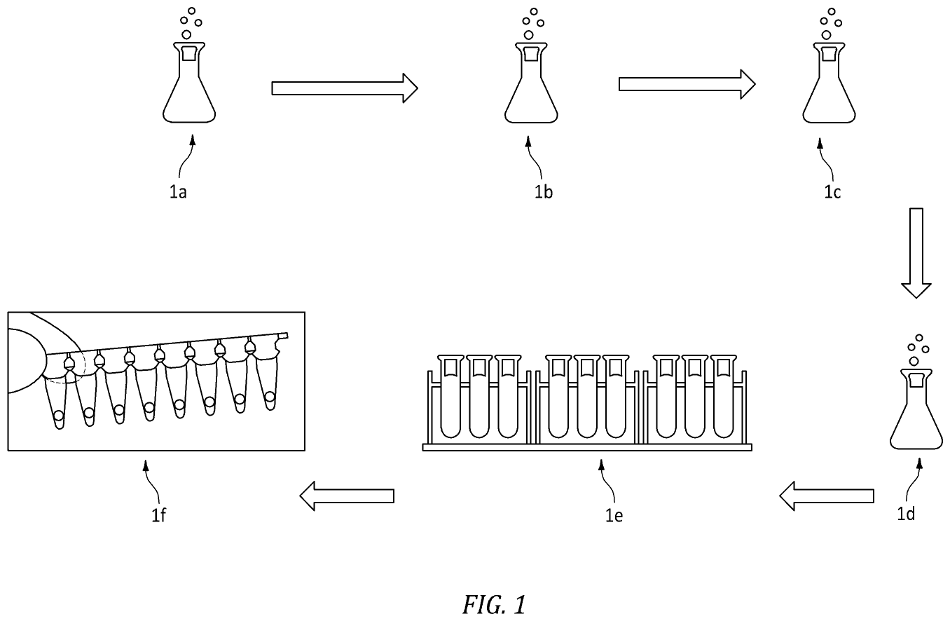

Turning to FIG. 1, an exemplary procedure for preparing an indicator master mix in the form of Calcein-containing LAMP reaction lyobeads is illustrated. In the illustrated embodiment, the procedure is for preparing about 500 units of lyobeads that are each about 25 uL in size. However, as may be recognized by those of ordinary skill in the pertinent art based on the teachings herein, any number of lyobeads in any size may be formed, and further, the lyophilizable or lyophilized indicator master mix may be provided in any other form factor, each of which as may be currently known or that later becomes known. As shown in FIG. 1, Step 1a is directed to creating the Calcein-DMSO mixture. In the exemplary embodiment, an approximately 50 millimole (mM) solution is prepared. Step 1a includes setting the Tare balance to zero, weighing out about 6 micrograms (ug) of Calcein and introducing the weighed amount of Calcein into a 1.7 mL tube. Then, about 0.193 uL of DMSO is added to the Calcein in the tube. The solution is mixed until the Calcein is substantially dissolved in the DMSO. Gentle stirring, agitation or other form of additional energy may be employed to facilitate dissolving the Calcein in the DMSO.

Step 1b is directed to preparing an approximately 20.4 mM MnCl₂ solution. The Tare balance is set to zero. Approximately 28 μg of MnCl₂ is weighed and introduced into a 15 mL tube. Approximately 6.94 mL of RNAse free water is added to the 1.5 mL tube to create a 20.4 mM concentration. The solution is mixed until the MnCl₂ is substantially or completely dissolved in the RNAse free water. Gentle stirring, agitation or other form of additional energy may be employed to facilitate dissolving the MnCl₂ in the RNAse water.

Step 1c is directed to creating a 40× Quenched Calcein (or “Quenched Indicator”) solution (40× Indicator). This step involves adding about 10 μL of the 50 mM Calcein-DMSO solution of Step 1a to about 490 μL of the 20.4 mM MnCl₂ solution of Step 1b to a 1.5 mL tube and allowing the solution to mix or facilitating the mixture, such as by gentle stirring, agitation or other form of additional energy.

Step 1d is directed to preparing an approximately 500 lyobead solution. This step involves combining the following ingredients in the amounts as follows:

| Ingredient(s) | Amounts |
|---|---|
| LAMP 4X Master Mix | about 3125 uL |
| LAMP Primer Mix (can vary but typically has | about 1250 uL |
| 2 forward, 2 backward and 1 loop primer) | |
| 40X Indicator prepared in Step 1c above | about 312.5 uL |
| Excipient(s) | about 2500 uL |
| Water | about 5312.5 uL |
| Total: | about 12,500 uL |

As indicated in FIG. 1, the LAMP Primer Mix can vary in a manner known to those of ordinary skill in the pertinent art, but typically has approximately two forward primers, two backward primers and one loop primer. The LAMP 4× Master Mix, LAMP Primer Mix and Excipient(s) may be any of numerous such products that are currently known and/or commercially available, or that later become known and/or commercially available. For example, the LAMP 4× Master Mix may be the “Lyo-Ready™ LAMP Mix, 4×” available from Meridian Bioscience, the “Polaris™ Lyophilizable LAMP Master Mix 4×” available from NZYtech located in Lisboa, Portugal, or the Custom WarmStart® LAMP 4× Master Mix from New England Biolabs. The LAMP primer mix may be any specific synthetic oligonucleotide consisting of forward, backward and loop primers. As indicated above, the Excipients are commercially available and are of a type known to those of ordinary skill in the pertinent art to facilitate the manufacture, processing and/or performance of the lyobeads. For example, the Excipient(s) may be sucrose. As may be recognized by those of ordinary skill in the pertinent art based on the teachings herein, the foregoing LAMP 4× Master Mix, LAMP Primer Mix and Excipient(s) and the respective amounts indicated are only exemplary, and numerous different such ingredients and/or amounts that are currently known, or that later become known, may be employed.

In Step 1e, the bead solution of Step 1d (e.g., 500 bead solution) is aliquoted into individual reaction tubes. In the exemplary embodiment, each aliquot is about 25 uL. However, as may be recognized by those of ordinary skill in the pertinent art based on the teachings herein, the size of each such unit may be changed as desired or required, and any desired or required number of units may be prepared.

In Step 1f, the aliquots in the individual reaction tubes are lyophilized until dry. In the exemplary embodiment, the lyophilization takes about 12 hours.

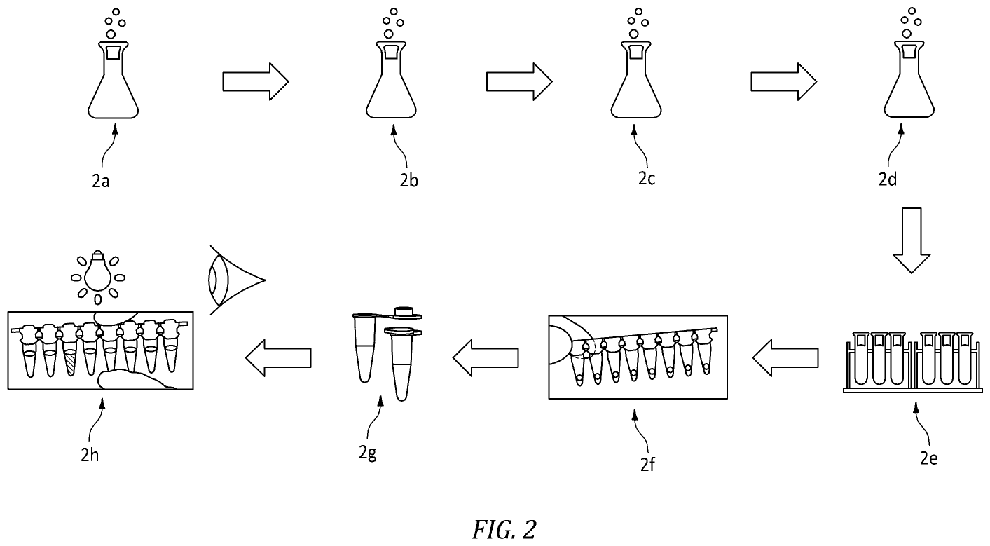

Turning to FIG. 2, the flowchart includes steps 2a though 2f that are the same as Steps 1a through if for preparing the lyobead master mix as set forth in FIG. 1, and further includes Steps 2g and 2h for rehydrating the lyobeads with processed biological samples and reading the results with a human eye. As shown in Step 2g, an approximately 25 uL biological sample processed in a manner known to those of ordinary skill in the pertinent art is added to a lyobead in a respective reaction tube to rehydrate the lyobead. The biological sample may be, for example, blood or saliva. The resulting mixture is then incubated for a substantially predetermined period of time at a substantially predetermined temperature. In the exemplary embodiment, the resulting mixture is incubated at about 60° C. for about 45 minutes. However, as may be recognized by those of ordinary skill in the pertinent art based on the teachings herein, this temperature and time are only exemplary, and the temperature(s) and time(s) or timing cycle may be changed as desired or otherwise required.

As shown schematically in FIG. 2, Step 2h is directed to reading the test results with a human eye. As also shown schematically in FIG. 2, the reaction tubes containing the incubated biological sample and master mix mixtures are illuminated with an excitation lamp. Since the master mix includes Calcein as the visual indicator, the reaction tubes are illuminated with fluorescent light. The tube(s) (PCR tubes in the illustrated embodiment) that glow green (or other distinguishing color) under the excitation lamp contain the target molecule, whereas those tubes that do not glow green (or other distinguishing color) do not contain the target molecule and thus indicate the absence of the target molecule. As shown schematically in FIG. 2, the distinguishing color or lack thereof, and thus the test results, can be identified with the human eye.

As may be recognized by those of ordinary skill in the pertinent art based on the teachings herein, the target molecule may be any desired target or template DNA or RNA sequence that can be targeted by nucleic acid amplification and visually identified, that is currently known, or that later becomes known. For example, in one embodiment of the present invention, the indicator master mix and methods are used to identify the gender of a fetus as disclosed, for example, in the following co-pending patent application assigned to the assignee of the present invention and hereby incorporated by reference in its entirety as part of the present disclosure: U.S. patent application Ser. No. 18/493,412 of Timothy Alcorn and Peter Latham, filed Oct. 23, 2023, entitled “Rapid, Microfluidic Diagnostic Device and Method For Biological Sex Determination,” published as U.S. Patent Publication No. US 2024/0131516 A1.

One advantage of the indicator master mix and methods of the present disclosure is that the indicator master mix is lyophilizable or is lyophilized and includes a detectable indicator/agent that is visibly detectable by a human eye. Such indicator master mixes can be formulated for LAMP, PCR or other nucleic acid amplification assay techniques. The lyophilized indicator master mix is advantageous because it has a relatively long shelf life, and therefore can be stored dry and later combined with reagent(s) and a biological sample, such as blood or saliva, to perform a nucleic acid amplification assay to detect and visually identify the presence or the absence of a target or template DNA or RNA sequence in the biological sample.

Yet another advantage of the indicator master mix and methods of the present disclosure is that they overcome the problems encountered in the above-described prior art of lyophilizing master mixes containing Calcein or other substantially hydrophobic nucleic-acid based indicators. As indicated above, the use of DMSO or other solvents has been required to solubilize the Calcein or other substantially hydrophobic nucleic-acid based indicator into master mix solutions. While the DMSO or other solvents have been able to effectively solubilize the Calcein or other substantially hydrophobic nucleic-acid based indicators, they have prevented lyophilization of the resulting solutions. Rather, attempts to lyophilize such solutions have resulted in the formation of sludge-like mixtures that cannot be lyophilized into a useful form factor. The master mix and methods of the present disclosure overcome this problem by sufficiently diluting out the DMSO or other solvent as compared to the Calcein or other substantially hydrophobic nucleic-acid based indicator. The resulting master mix exhibits a surprisingly effective balance between the relative amount of Calcein or other substantially hydrophobic nucleic-acid based indicator as compared to the amount of DMSO or other solvent to both solubilize the indicator into solution with the solvent while nevertheless creating a solution that can be lyophilized (or dehydrated) and provided in a useful form factor, such as a lyobead. On the one hand, the master mix has a sufficient amount of DMSO or other solvent as compared to the amount of Calcein or other substantially hydrophobic nucleic-acid based indicator to solubilize the Calcein or other substantially hydrophobic nucleic-acid based indicator into solution with the solvent. The solubilization of the Calcein or other substantially hydrophobic nucleic-acid based indicator in the DMSO or other solvent can occur naturally or, if necessary, with the assistance of additional energy, such as stirring or agitation. On the other hand, the relative amount of DMSO or other solvent is sufficiently diluted as compared to the amount of Calcein or other substantially hydrophobic nucleic-acid based indicator to allow the solution to be lyophilized and provided in a useful form factor. Accordingly, in the indicator master mix and methods of the present disclosure, the relative amount of nucleic-acid based indicator to solvent is preferably within the range of (i) about 0.01 gm nucleic-acid based indictor/gm solvent to (ii) about 0.05 gm nucleic-acid based indicator/gm solvent, and more preferably, is within the range of (i) about 0.009 gm nucleic-acid based indictor/gm solvent to (ii) about 0.045 gm nucleic-acid based indicator/gm solvent. Such relative amount of Calcein or other substantially hydrophobic nucleic-acid based indicator to DMSO or other solvent surprisingly and unexpectedly achieves dissolution or solubilization of the Calcein or other substantially hydrophobic nucleic-acid based indicator into solution with the DMSO or other solvent, while nevertheless providing an indicator master mix that is lyophilizable or lyophilized in a useful form factor. Although in the exemplary embodiments the form factor of the indicator master mix is a lyobead, the lyophilized master mix may take any of numerous other form factors that are currently known, or that later become known, such as a cake or a loose powder.

As may be recognized by those of ordinary skill in the pertinent art based on the teachings herein, numerous changes, modifications and additions may be made to the above-described and other embodiments of the present invention without departing from the scope of the invention. For example, the formulations, ingredients, components and methods of operation and use, may take any of numerous different forms or configurations, and may be made of or use any of numerous different materials, components or ingredients, that are currently known or that later become known, and features or aspects may be added or removed, without departing from the scope of the invention. This detailed description of embodiments is therefore to be taken in an illustrative as opposed to a limiting sense.

What is claimed is:

1. An indicator master mix for performing a nucleic acid amplification assay to detect and visually identify a presence or an absence of a target or template DNA or RNA sequence, comprising: a human readable, substantially hydrophobic nucleic-acid based indicator, and a solvent configured to solubilize the substantially hydrophobic nucleic-acid based indicator, wherein the relative amount of nucleic-acid based indicator to solvent is within the range of (i) about 0.01 gm nucleic-acid based indictor/gm solvent to (ii) about 0.05 gm nucleic-acid based indicator/gm solvent, wherein the solvent solubilizes the substantially hydrophobic nucleic-acid based indicator, the indicator master mix is configured to perform the nucleic acid amplification assay to detect and visually identify the presence or the absence of the target or template DNA or RNA sequence, and the substantially hydrophobic nucleic-acid based indicator is visually detectable by a human eye to indicate the presence or absence of the target or template DNA or RNA sequence.

2. An indicator master mix as defined in claim 1, wherein the human readable, substantially hydrophobic nucleic-acid based indicator is configured to react to one or more diprotic cations to detect and visually identify the presence of the target or template DNA or RNA sequence.

3. An indicator master mix as defined in claim 1, wherein the human readable, substantially hydrophobic nucleic-acid based indicator is isothermal.

4. An indicator master mix as defined in claim 1, wherein the relative amount of nucleic-acid based indicator to solvent is within the range of (i) about 0.009 gm nucleic-acid based indictor/gm solvent to (ii) about 0.045 gm nucleic-acid based indicator/gm solvent.

5. An indicator master mix as defined in claim 1, wherein the human readable, substantially hydrophobic nucleic-acid based indicator is quenched Calcein.

6. An indicator master mix as defined in claim 5, wherein the solvent is an aprotic solvent.

7. An indicator master mix as defined in claim 6, wherein the aprotic solvent is selected from the group including: Acetone, Acetonitrile, DMF (N,N-Dimethylformamide), DMSO (Dimethyl sulfoxide), DCM (Dichloromethane), THF (Tetrahydrofuran), HMF (hydroxymethylfurfural) and Crown ethers.

8. An indicator master mix as defined in claim 7, wherein the aprotic solvent is DMSO.

9. An indicator master mix as defined in claim 1, wherein the solvent is configured to solubilize the substantially hydrophobic nucleic-acid based indicator into an aqueous-based solution of the indicator master mix.

10. An indicator master mix as defined in claim 1, wherein the indicator master mix is substantially dehydrated and defines a form factor configured to perform a nucleic acid amplification assay on a respective biological sample to detect and visually identify the presence of a target or template DNA or RNA sequence in the respective biological sample.

11. An indicator master mix as defined in claim 10, wherein the indicator master mix is lyophilized.

12. An indicator master mix as defined in claim 11, wherein the indicator master mix is formed into a plurality of lyobeads, and one or more of the lyobeads beads are configured to perform a nucleic acid amplification assay on a respective biological sample.

13. An indicator master mix for performing a nucleic acid amplification assay to detect and visually identify a presence or an absence of a target or template DNA or RNA sequence in a biological sample, comprising: first substantially hydrophobic means for detecting and visually identifying with a human eye the presence or the absence of the target or template DNA or RNA sequence in the biological sample; and second means for solubilizing the first means, wherein the relative amount of first means to second means is within the range of (i) about 0.01 gm first means/gm second means to (ii) about 0.05 gm first means/gm second means, the indicator master mix is configured to perform the nucleic acid amplification assay to detect and visually identify the presence or the absence of the target or template DNA or RNA sequence in the biological sample, and the first means is visually detectable by the human eye to indicate the presence or the absence of the target or template DNA or RNA sequence in the biological sample.

14. An indicator master mix as defined in claim 13, wherein the first means is a human readable, substantially hydrophobic nucleic-acid based indicator, and the second means is a solvent configured to solubilize the substantially hydrophobic nucleic-acid based indicator.

15. An indicator master mix as defined in claim 14, wherein the human readable, substantially hydrophobic nucleic-acid based indicator is quenched Calcein, and the solvent is an aprotic solvent selected from the group including: Acetone, Acetonitrile, DMF (N,N-Dimethylformamide), DMSO (Dimethyl sulfoxide), DCM (Dichloromethane), THF (Tetrahydrofuran), HMF (hydroxymethylfurfural) and Crown ethers.

16. A method comprising the following steps:
solubilizing a human readable, substantially hydrophobic nucleic-acid based indicator in a solvent, wherein the relative amount of nucleic-acid based indicator to solvent is within the range of (i) about 0.01 gm nucleic-acid based indictor/gm solvent to (ii) about 0.05 gm nucleic-acid based indicator/gm solvent;
combining the nucleic-acid based indicator and solvent solution with an aqueous-based solution containing a cofactor or diprotic cation containing compound and quenching the nucleic-acid based indicator in the solution; and
lyophilizing the quenched nucleic-acid based indicator solution and forming a lyophilized master mix wherein the relative amount of nucleic-acid based indicator to solvent in the lyophilized master mix is within the range of (i) about 0.01 gm nucleic-acid based indictor/gm solvent to (ii) about 0.05 gm nucleic-acid based indicator/gm solvent.

17. A method as defined in claim 16, further comprising adding to the quenched nucleic-acid based indicator solution one or more enzymes, nucleotides, buffers, stabilizers, enhancers, primers, probes and excipients, prior to the step of lyophilizing.

18. A method as defined in claim 17, further comprising aliquoting the master mix into a plurality of reaction vessels prior to the step of lyophilizing.

19. A method as defined in claim 18, wherein the reaction vessels are reaction tubes, and at least a portion of each reaction tube is transparent or substantially transparent to enable visualization therethrough by a human eye to visually observe a presence or an absence of the target or template DNA or RNA sequence.

20. A method as defined in claim 16, wherein the human readable, substantially hydrophobic nucleic-acid based indicator is Calcein, and the cofactor or diprotic cation containing compound is manganese chloride (MnCl₂).

21. A method comprising the following steps:
hydrating a lyophilized indicator master mix with a processed biological sample, wherein the lyophilized master mix contains a quenched nucleic-acid based indicator and a solvent and the relative amount of nucleic-acid based indicator to solvent is within the range of (i) about 0.01 gm nucleic-acid based indictor/gm solvent to (ii) about 0.05 gm nucleic-acid based indicator/gm solvent; and
visually observing the appearance of the hydrated master mix and biological sample with a human eye and detecting with the human eye a presence or an absence of a target or template DNA or RNA sequence.

22. A method as defined in claim 21, further comprising incubating the hydrated master mix and biological sample at a substantially constant temperature for a substantially predetermined period of time prior to visually observing the appearance of the hydrated master mix and biological sample and then visually detecting the presence or the absence of the target or template DNA or RNA sequence.

23. A method as defined in claim 22, wherein the human readable, substantially hydrophobic nucleic-acid based indicator is Calcein, and the solvent is selected from the group including Acetone, Acetonitrile, DMF (N,N-Dimethylformamide), DMSO (Dimethyl sulfoxide), DCM (Dichloromethane), THF (Tetrahydrofuran), HMF (hydroxymethylfurfural) and Crown ethers.
